(19) United States
(12) Patent Application Publication
(10) Pub. No.: US 2026/0232272 A1
(43) Pub. Date: Aug. 13, 2026

(54) SYSTEMS, METHODS, AND DEVICES TO MEASURE, MONITOR, AND ANALYZE BRAIN ACTIVITY AND BRAIN PRESSURE

(71) Applicant: CRAINIOSMART LLC, Mandeville, LA (US)

(72) Inventors: Ravi KANAGALA, Mandeville, LA (US); Frederick J. THABET, Boca Raton, FL (US)

(21) Appl. No.: 19/156,285

(22) PCT Filed: Feb. 9, 2024
(86) PCT No.: PCT/US2024/015114

§ 371 Date: Aug. 13, 2025

Related U.S. Application Data
(60) Provisional application No. 63/445,064, filed on Feb. 13, 2023.
(60) Provisional application No. 63/491,638, filed on Mar. 22, 2023.

(51) Int. Cl. A61B 5/00; A61B 5/03; A61B 5/0538; A61B 90/00

(52) U.S. Cl. CPC A61B 5/6853; A61B 5/031; A61B 5/036; A61B 5/0538; A61B 90/06; A61B 2090/061

(57) ABSTRACT

Systems, methods, and devices include a cranial pressure monitoring system (CPMS) with a sensor delivery system. The CPMS includes internal monitoring system to detect changes in intracranial pressure either acutely or chronically over time. The system is insertable through the nasal cavity to place a pressure monitor, such as an impedance monitoring device, (e.g., and/or other sensor(s)) in the region of the cribriform plate and/or the nasal concha (superior). As such, brain measurements can be taken through the thin bone material of the cribriform plate using non-invasive techniques.

### CROSS-REFERENCE TO RELATED APPLICATION

This application claims priority to U.S. Provisional Patent Application Ser. No. 63/445,064, filed Feb. 13, 2023 and titled “SYSTEMS, METHODS, AND DEVICES TO MEASURE, MONITOR, AND ANALYZE BRAIN ACTIVITY AND BRAIN PRESSURE;” and to U.S. Provisional Patent Application Ser. No. 63/491,638, filed Mar. 22, 2023 and titled “SYSTEMS, METHODS, AND DEVICES TO MEASURE, MONITOR, AND ANALYZE BRAIN ACTIVITY AND BRAIN PRESSURE,” the entireties of which are herein incorporated by reference.

### BACKGROUND

Cognitive characteristics and brain-related parameters are typically measured using invasive techniques (e.g., brain surgery) or with a sensor placed on the exterior of the cranium. As such, high-fidelity brain measurements are difficult to collect quickly and without specialized clinical equipment and personnel.

It is with these observations in mind, among others, that various aspects of the presently disclosed technology were conceived and developed.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system for measuring, monitoring, and/or analyzing brain activity and/or pressure with a brain measurement device having a sensor placement system;

FIG. 2 illustrates an example system for measuring, monitoring, and/or analyzing brain activity and/or pressure using a sensory delivery procedure and/or a sensor expansion procedure, which can form at least a part of the system depicted in FIG. 1; and

FIG. 3 illustrates an example method for measuring, monitoring, and/or analyzing brain activity and/or pressure, which can be performed by any of the systems depicted in FIGS. 1 and 2.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present technological concepts.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present concept be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present concept, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50°includes all values from 16.6° to 150°.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein include a cranial pressure monitoring system (CPMS) with a sensor delivery system to address the aforementioned problems. The CPMS can be a unique internal monitoring system to detect changes in intracranial pressure either acutely or chronically over time. The system can be easily inserted through the nasal cavity to place a pressure monitor, such as an impedance monitoring device, (e.g., and/or other sensor(s)) in the region of the cribriform plate and/or the nasal concha (superior). As such, brain measurements can be taken through the thin bone material of the cribriform plate quickly and easily using non-invasive techniques.

It is to be understood that the techniques disclosed herein can be used for any type of intracranial monitoring system which uses the nasal cavity for insertion of a sensor. Additionally or alternatively, the techniques disclosed herein can be used for placement of sensors at any portion of the whole cribriform plate area (e.g., the whole cribriform plate are or a sub-section of the cribriform plate area) to monitor any type of pressure (e.g., brain pressure), electrical impedance, and/or other bio-signals from this particular area of the body.

Additional advantages of the systems, methods, and devices discussed herein will become apparent from the detailed description below.

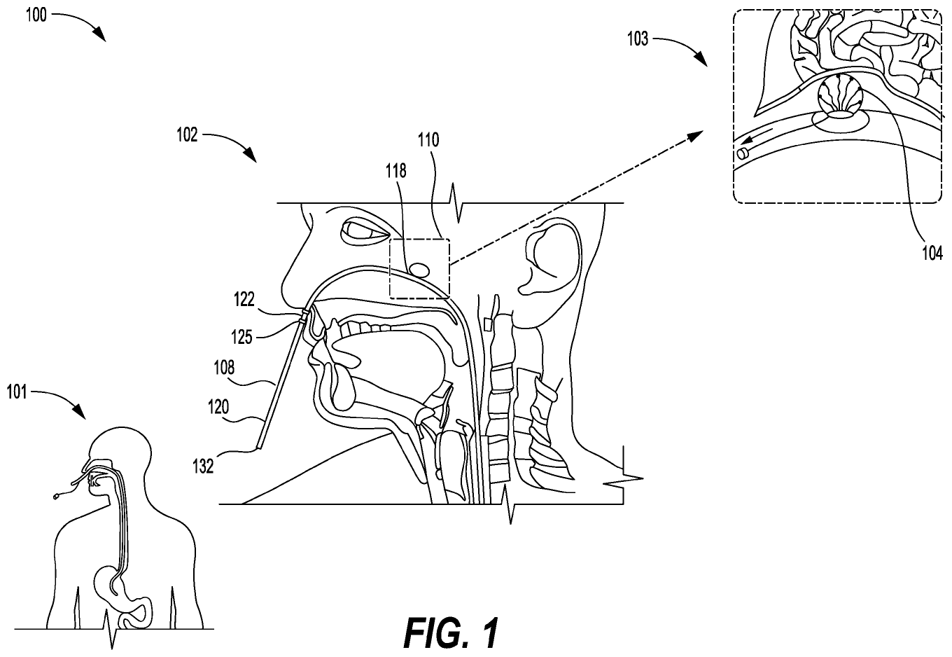

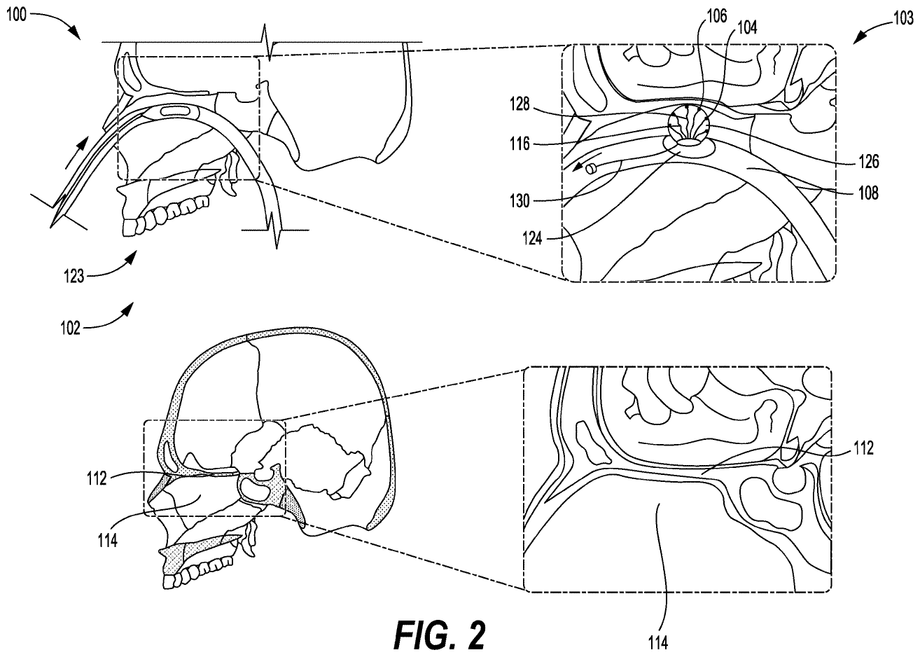

FIGS. 1 and 2 illustrate an example system 100 including a sensor placement system 102 for delivering one or more sensors 104 of a brain activity and/or pressure measurement device 103 to a cranial measurement location 106 of a patient 101. For instance, a nasogastric tube 108 (e.g., for being inserted through the nasal, and into the esophagus and stomach) can have an integrated delivery system 110 configured to place the sensor(s) 104 at the cribriform plate 112 and/or the superior nasal concha 114 using a sensor balloon 116. The sensor balloon 116 can be disposed in the nasogastric tube 108 at a predetermined location 118, which can be a predetermined distance 120 from an end of the nasogastric tube 108 and/or an intermediate distance location 122 (e.g., at a nasal entry location on the nasogastric tube 108). Once a delivery procedure 123 is performed to deliver and position the nasogastric tube 108 and the sensor balloon 116 at the target location (e.g., at the cribriform plate 112 and/or the superior nasal concha 114), a sensor release procedure can be performed by rotating the nasogastric tube 108. The rotation can cause a slit or opening 124 in the nasogastric tube 108 to face upward and/or towards the cribriform plate 112 and/or the superior nasal concha 114. Rotating the nasogastric tube 108 can expose the slit/opening 124 (e.g., an oval or circular opening) to the concave curvature of the cribriform plate 112, giving the sensor balloon 116 space to expand into and press against the cribriform plate 112. For instance, the sensor balloon 116 can be compressed, spring-loaded, or otherwise collapsed in a tensioned-manner such that it can expand automatically responsive to the rotation. Additionally or alternatively, the sensor balloon 116 can be fluidly coupled to a pump or wire to cause the expansion of the sensor balloon 116. Moreover, the sensor balloon 116 can include one or more rigid or semi-rigid structural components or ribs to expand the sensor balloon 116 and/or maintain a shape of the sensor balloon 116.

In some examples, the brain activity and/or pressure measurement device 103 can include one or more distance indicators 125 on an exterior surface of the nasogastric tube 108. The one or more distance indicators 125 can indicate the predetermined distance 120 such that the nasogastric tube 108 can be inserted a precise length into the nasal passageway in order to align the sensor balloon 116 with the cribriform plate 112 and/or the superior nasal concha 114. In some instances, the one or more distance indicators 125 can align with a nasal opening to indicate that the nasogastric tube 108 is inserted the predetermined distance 120 into the patient.

In some scenarios, the sensor(s) 104 can measure an impedance and/or a brain waveform indicating a brain pressure and/or brain function (e.g., pulsatile waveforms). The sensor balloon 116 can be a mesh and/or rubber material and can include the sensor(s) 104 disposed on its outer surface 126. The sensor(s) 104 can include at least one of a pressure sensor, (e.g., a thin-film pressure sensor, pulmonary artery pressure sensor, and/or any other type of pressure sensor), an electrical impedance sensor, an infrared sensor, an electroencephalography (EEG) sensor, an acoustic sensor, a motion sensor, a temperature sensor, combinations thereof, and so forth. The sensor balloon 116 can contact the cribriform plate 112 and/or the superior nasal concha 114 with substantial surface area such that multiple sensors 104 can be positioned against this thin bone to generate sensor data through the cribriform plate. This sensor data from multiple data collection points can be aggregated or averaged together. Furthermore, one or more wires 128 can connect to the sensor(s) 104 and can extend back into the nasogastric tube 108 and out the nasal to connect to a controller. For instance, the one or more wires 128 can be embedded or at least partially embedded in a material forming the sensor balloon 116. Once the sensor expansion process is performed, the sensor locations can be adjusted with minor adjustments to the nasogastric tube 108 (e.g., rotating, pushing, or pulling the nasogastric tube 108), for instance, until a clean data reading from the sensor(s) 104 is established.

In some examples, the sensor(s) 104 can include a pH sensor (e.g., a combination pH sensor, a differential pH sensor, etc.) for measuring a pH value in the nasal cavity, such as at the cribriform plate 112 and/or the superior nasal concha 114. The device can use the pH sensor for detecting a pH value which can be used for identifying the reason a person falls unconscious. For instance, a person can become unconscious for many reasons, such as stroke or dehydration. Some causes for becoming unconscious can affect the alkalinity/acidity of the body fluids within the nasal cavity. An extreme gastric issue can cause a parasympathetic response which results in the person losing consciousness. A low pH measurement using the technology disclosed herein can indicate a high acidity resulting from the extreme gastric issue, such that the extreme gastric issue can be identified as the cause for becoming unconscious. Furthermore, a pulmonary embolism can also cause a person to lose consciousness. In this case, the person can stop breathing, and this failure to release the CO₂ from their lungs can cause their pH level to drop. As such, a low pH measurement using this technology can also be used to identify a pulmonary embolism as the cause for losing consciousness.

In some instances, a data collection and analysis procedure can be performed with the data collected by the sensor(s) 104. The sensor(s) 104 can communicate the sensor data and/or the health parameters to one or more controller devices (e.g., computer(s) including a processor, a memory storing executable instructions, and a display) using various wired or wireless connections (e.g., Bluetooth). Useful brain-related health parameters, such as brain waveforms and/or electrical signals, can be collected, presented, and assessed in real-time. Brain-related diagnosis can be made using the sensor placement system 102 within seconds of inserting the nasogastric tube 108. Measured values can be presented as graphical indications (e.g., icons, alphanumeric symbols, graphs, images, sounds, etc.) and/or with indications of whether the measured values are above or below a predetermined threshold. Once the data collection and/or analysis procedure is complete, a removal procedure can be performed to extract the sensor balloon 116 from the target measurement area (e.g., the cribriform plate 112 and/or the superior nasal concha 114). A rope, a wire, a rod, or other type of elongated flexible or rigid material (e.g., hereafter referred to as a “cord 130”), and/or a pulley system can attach to the sensor balloon 116, pass through the opening 124, and out the end 132 of the nasogastric tube 108. Pulling the cord 130 can cause the sensor balloon 116 to collapse back into the opening 124 and the nasogastric tube 108. The cord 130 can also be pulled and/or adjusted to reposition the sensor(s) 104. For instance, the sensor balloon 116 can be retracted back into the nasogastric tube 108 for positioning the sensor balloon 116 and/or for removing the sensor balloon 116. In some instances, the sensor balloon 116 can form a soft sheath surrounding the sensor(s) 104 and/or surround rough edges of the brain activity and/or pressure measurement device 103 such that the expansion and the collapsing of the sensor balloon 116 does not irritate or damage the soft tissue around the target delivery area.

In this way, characteristics of the patient's brain can be measured, monitored, and analyzed quickly and without requiring any incisions or surgery. As such, the brain activity and/or pressure measurement device 103 can be used out in the field, at sports events, schools, offices, and other non-clinical environments. Additionally or alternatively, the brain activity and/or pressure measurement device 103 can be used in a clinic setting to monitor and assess brain-related characteristics of the patient (e.g., in clinical office, an urgent care room, an operating room and/or an emergency room). The brain activity and/or pressure measurement device 103 can be Collected data can include brain waveforms, electrical signals, pressure/impedance readings, and/or other types of data. The data can be collected, presented, and/or assessed in real-time to provide a real-time measurement of brain activity/pressure or other parameters.

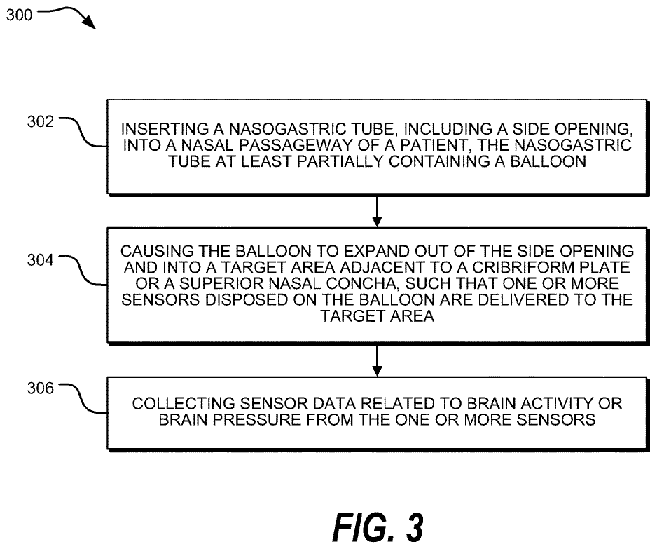

Turning to FIG. 3, an example method 300 for measuring, monitoring and/or analyzing brain activity, brain pressure, and/or other brain-related parameters. The method depicted in FIG. 3 can be performed by the systems and devices depicted in FIGS. 1 and 2.

In some examples, at operation 302, the method 300 inserts a nasogastric tube, including a side opening, into a nasal passageway of a patient, the nasogastric tube at least partially containing a balloon. At operation 304, the method can cause the balloon to expand out of the side opening and into a target area adjacent to a cribriform plate or a superior nasal concha, such that one or more sensors disposed on the balloon are delivered to the target area. At operation 306, the method 300 can include collecting sensor data related to brain activity or brain pressure from the one or more sensors.

It is to be understood that the specific order or hierarchy of steps in the method(s) depicted in FIG. 3 and throughout this disclosure are instances of example approaches and can be rearranged while remaining within the disclosed subject matter. For instance, any of the operations depicted in FIG. 3 and throughout this disclosure may be omitted, repeated, performed in parallel, performed in a different order, and/or combined with any other of the operations depicted in FIG. 3 and throughout this disclosure.

While the presently disclosed technology has been described with reference to various implementations, it will be understood that these implementations are illustrative and that the scope of the presently disclosed technology is not limited to them. Many variations, modifications, additions, and improvements are possible. More generally, implementations in accordance with the presently disclosed technology have been described in the context of particular implementations. Functionality may be separated or combined differently in various implementations of the disclosure or described with different terminology. These and other variations, modifications, additions, and improvements may fall within the scope of the disclosure as defined in the claims that follow.

1. A brain measurement and analysis device including:
a tube with a side opening a predetermined distance from:
an end of the tube; or
an intermediate location on the tube;
a balloon at least partially positionable within the tube at the side opening and including one or more sensors, the balloon being configured to:
expand out of the side opening as part of a sensor placement procedure; and
retract into the side opening as part of a sensor withdrawal procedure; and
a cord coupled to the balloon and passing at least partially through an inner channel of the tube.

2. The brain measurement device of claim 1,
wherein,
the one or more sensors include a plurality of sensors embedded in the balloon.

3. The brain measurement device of claim 2,
further comprising,
a plurality of wires connected to the plurality of sensors, embedded in the balloon, and extending along an inner channel of the tube to couple the plurality of sensors to a controller outside a patient receiving the one or more sensors.

4. The brain measurement device of claim 1,
wherein,
the cord is configured to pull the balloon through the side opening back into the tube responsive to a force applied to the cord.

5. The brain measurement device of claim 1,
wherein,
the balloon is configured to automatically expand using a release of a compression of material forming the balloon.

6. The brain measurement device of claim 1,
wherein the one or more sensors include an impedance sensor.

7. The brain measurement device of claim 6,
wherein,
the one or more sensors include a pressure sensor.

8. The brain measurement device of claim 1,
wherein,
the tube is a nasogastric tube with an exterior having one or more distance indicators corresponding to a distance from a nasal opening to a cribriform plate or a superior nasal concha.

9. The brain measurement device of claim 8,
wherein,
the side opening is a circular opening or an oval opening.

10. The brain measurement device of claim 1,
wherein,
the balloon is formed of a flexible mesh material.

11. A brain measurement system including:
a nasogastric tube with a side opening;
one or more visual indicators disposed on the nasogastric tube a predetermined distance from the side opening;
a balloon having one or more sensors embedded into a material forming the balloon, the balloon being expandable out the side opening to place the one or more sensors at a target location, and at least partially retractable back into the side opening;
one or more sensors disposed on a sensor balloon, the sensor balloon being positionable within the nasogastric tube and configured to:
expand out of the side opening as part of a sensor placement procedure; and
retract into the side opening as part of a sensor withdrawal procedure.

12. The brain measurement system of claim 11,
further including,
a cord disposed in the nasogastric tube coupled to the balloon for pulling the balloon back into the side opening.

13. The brain measurement system of claim 11,
wherein,
the balloon has a shape configured to conform to a contour of a cribriform plate or a superior nasal concha.

14. A method of brain measurement, the method including:
inserting a nasogastric tube, including a side opening, into a nasal passageway of a patient, the nasogastric tube at least partially containing a balloon;
causing the balloon to expand out of the side opening and into a target area adjacent to a cribriform plate or a superior nasal concha, such that one or more sensors disposed on the balloon are delivered to the target area; and
collecting sensor data related to brain activity or brain pressure from the one or more sensors.

15. The method of claim 14,
further including,
determining a brain-related anatomical characteristic based on the sensor data; and
present one or more indications of the brain-related anatomical characteristic at a display of a computing device.

16. The method of claim 14,
further comprising,
rotating the nasogastric tube to reposition the side opening and direct the side opening towards the cribriform plate or the superior nasal concha.

17. The method of claim 16,
wherein,
rotating the nasogastric tube includes rotating the nasogastric tube between 45 and 180 degrees.

18. The method of claim 14,
further including,
prior to inserting the nasogastric tube, placing the balloon into the side opening with a cord coupled to the balloon extending through the nasogastric tube and out an end of the nasogastric tube.

19. The method of claim 14,
further including,
removing the balloon from the target area by applying a force to a cord coupled to the balloon and passing through an inner channel of the nasogastric tube.

20. The method of claim 14,
wherein,
the one or more sensors include plurality of sensors; and
causing the balloon to expand causes at least one of the plurality of sensors to form contact with the cribriform plate or the superior nasal concha.
